(19) United States
(12) Patent Application Publication
(10) Pub. No.: US 2021/0118537 A1
(43) Pub. Date: Apr. 22, 2021

(54) SYSTEM AND METHOD FOR SENSOR-BASED HEALTHCARE MANAGEMENT

(71) Applicant: TAHANI A.H.B.A. ALHAJERI, SAFAT (KW)

(72) Inventor: TAHANI A.H.B.A. ALHAJERI, SAFAT (KW)

(21) Appl. No.: 16/656,527

(22) Filed: Oct. 17, 2019

(51) Int. Cl. G16H 20/10; A61B 5/00; A61K 36/48; A61K 33/38; G16H 80/00

(52) U.S. Cl. CPC G16H 20/10; A61B 5/0026; A61B 5/443; A61K 9/51; A61K 36/48; A61K 33/38; G16H 80/00; A61B 5/445

(57) ABSTRACT

The system and method for sensor-based healthcare management may provide a patch capable of accepting multiple individual biosensors of a health care professional's choosing for monitoring a patient's condition. A patch may be prepared by attaching the prescribed sensors and then applied to the affected tissue. A reader may receive data transmitted wirelessly from the sensors and forward the data to a remote location through a communications network. The monitoring health care professional at the remote location may then prescribe a patch having a different array of sensors, or prescribe an updated healthcare plan based on the received data.

### 1. FIELD OF THE INVENTION

The disclosure of the present patent application relates to healthcare systems, and more particularly to a system and method for sensor-based healthcare management that provides a system of remote biosensors for sensing multiple physiological conditions, reporting the sensed data to a practitioner over a network, and pads for dispensing nanoparticle treatments selected by the practitioner.

### 2. DESCRIPTION OF THE RELATED ART

Home-based healthcare provides an inexpensive solution for many conditions in the field of healthcare. Many conditions require frequent trips to a medical professional for treatment. In many cases, it may be difficult for the patient to make the trips. This is especially the case for the disabled and elderly. In addition, each trip can cost a considerable amount of money to the patient. Thus, obviating the need for frequent trips to the medical professional will significantly improve the patient's quality of life.

A variety of home-based healthcare plans for different diseases are known in the field. However, it will be clear to one of skill in the medical arts that all these healthcare plans monitor the levels of a certain set of conditions related to the disease and fail to provide the medical professional with the flexibility to choose and change which disease-related conditions to be detected according to the health state of the patient.

Biosensors have gained great interest globally, as they provide a convenient way to quantitatively convert information about the presence of chemical and biological species into a measurable, useful signal. Generally, the basic components of any biosensor comprise: (1) the bio-recognition receptor that interacts with the biomolecule of interest; (2) the transducer that converts the information into a measureable signal; and (3) the electronic system, which includes signal amplifier, processor, and display.

Wound healing is an innate mechanism of action that works reliably most of the time. A key feature of wound healing is stepwise repair of lost extracellular matrix (ECM) that forms the largest component of the dermal skin layer. As a result of diabetes mellitus, diabetic foot ulcers takes place as a result of various factors, such as mechanical changes in conformation of the bony architecture of the foot, peripheral neuropathy, and atherosclerotic peripheral arterial disease. A diabetic foot ulcer is caused by neuropathic (nerve) and vascular (blood vessel) complications of diabetes. Ulceration is the outcome of the lack of healthy blood flow. Untreatable infections resulting from ulcers may require amputations, or can take several weeks to heal. Additionally, ulcers may take longer to heal if the patient's blood sugar is high and if constant pressure is applied to the ulcer. In people who have good circulation and good medical care, an ulcer sometimes can heal in as few as three to six weeks, with deeper ulcers taking 12 to 20 weeks.

Ulcers and other medical conditions of the skin may require constant monitoring to insure optimum healing conditions. Accordingly, a healthcare management system and method that provides constant at-home monitoring of tissue surrounding a medical condition is desired.

Thus, a system and method for sensor-based healthcare management solving the aforementioned problems is desired.

### SUMMARY OF THE INVENTION

The system and method for sensor-based healthcare management may provide a patch capable of accepting multiple individual sensors of a health care professional's choosing. The patch may include therapeutic nanoparticles to assist in healing a covered wound, sore, or medical condition. Multiple sensors strips, capable of attachment to the patch, may each provide feedback directed at different physiological characteristics, such as pH, moisture, temperature, and where there is risk of microbial infection, bacterial amounts and bacterial types. Each sensor may include an inductive coil for wirelessly communicating with a reader. The reader may convert the raw data to readable metrics or medical information for different disease-related conditions. The processed data may then be sent to a practitioner over a network.

The method of providing sensor-based healthcare management may include prescribing an array of sensors to monitor a wound or sore of a patient; preparing a patch by attaching the prescribed sensors and applying the patch to the wound or sore; receiving data from the sensors and transmitting the data to a remote location; and prescribing a patch having a different array of sensors, or prescribing an updated healthcare plan, based on the received data.

These and other features of the present disclosure will become readily apparent upon further review of the following specification and drawings.

### BRIEF DESCRIPTION OF THE DRAWINGS

FIG. 1 is a diagrammatic top view of an electronic nano-pad or patch that may be used to implement a system for sensor-based healthcare management.

FIG. 2 is a diagrammatic side view of the electronic nano-pad or patch of FIG. 1.

FIG. 3A is a diagrammatic perspective view of a biosensor that may be used to implement a system for sensor-based healthcare management.

FIG. 3B is a detail view of area 3B, shown in FIG. 3A.

FIG. 3C is a top view of the biosensor of FIG. 3A.

FIG. 4 is a simplified block diagram of a system for sensor-based healthcare management.

FIGS. 5A 5B, and 5C are SEM micrographs of chlorophyll nanoparticles used in treatment patches in a system and method for sensor-based healthcare management, shown at different magnifications.

Similar reference characters denote corresponding features consistently throughout the attached drawings.

### DETAILED DESCRIPTION OF THE PREFERRED EMBODIMENTS

The system and method of sensor-based healthcare management may provide a patch capable of accepting multiple individual sensors of a health care professional's choosing. The patch may include therapeutic nanoparticles to assist in healing a covered wound, sore, or medical condition. Multiple sensors strips, capable of attachment to the patch, may each provide feedback directed at a different physiological characteristics, such as pH, moisture, temperature, and where there is risk of microbial infection, bacterial amounts and bacterial types. Each sensor may include an inductive coil for wirelessly communicating with a reader. The reader may convert the raw data to readable metrics or medical information for different disease-related conditions. The processed data may then be sent to a health care practitioner over a network.

The method for sensor-based healthcare management may include prescribing an array of sensors to monitor a wound or sore of a patient; preparing a patch by attaching the prescribed sensors and applying the patch to the wound or sore; receiving data from the sensors and transmitting the data to a remote location; and prescribing a patch having a different array of sensors or prescribing an updated healthcare plan based on the received data.

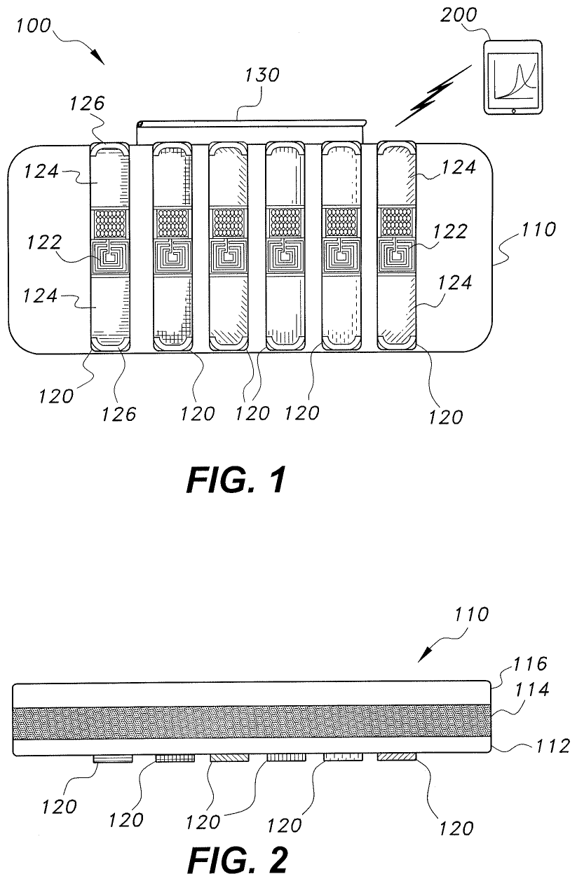

FIG. 1 shows an internal surface of a patch 110 with attached sensor strips 120. Each sensor strip 120 may contain a single sensor 122 configured to detect a different, specific physiological characteristic. For example, in the embodiment shown in FIG. 1, the array of strips 120 may be designed to sense pH, humidity, temperature, glucose, bacterial species, and bacterial count. In some embodiments, a portion, or all, of the sensors 122 may be biosensors. The strips 120 may have indicators, such as color coding, to indicate which physiological characteristic each strip 120 is configured to monitor. The sensor strips 120 may extend along a length of the patch 110 and have a loop 126 on one or both ends. The loop 126 may be used for attachment and removal, as well as indicating the type of sensor 122 to a user when the internal surface of the patch is facing down when attached to a patient. A roll 112 of bandage or adhesive material, having a length and width greater than the patch 130, may be placed over the patch 110 to secure the patch 110 to the patient and protect the environment around a wound or sore being monitored by the patch 110.

A reader 200 may be used to acquire raw data from the sensors 122, process the raw data into usable metrics, and transmit the usable metrics to a remote location over a network. The sensors 122 may be passive sensors that are powered through inductive coupling, similar to an RFID system. Accordingly, the reader 200 may produce an electromagnetic field by periodically polling the sensors 121 that induces an electric current in the sensors 121. With an electric current running therethrough, the sensors 121 can transmit data, which the reader 200 receives.

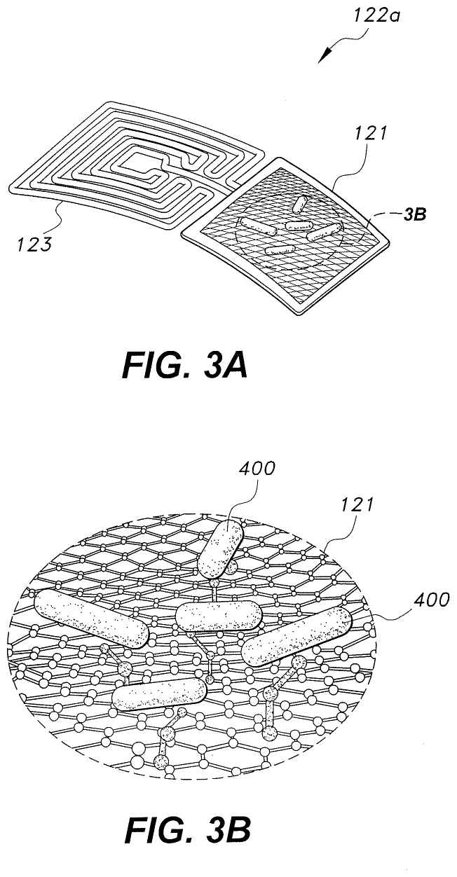

FIG. 2 shows a side view of an embodiment of the patch 110. The patch 110 may include three layers, an internal layer 112 containing silicon, a central porous layer 114, and an external layer 116, which may include therapeutic agents. Each layer 112, 114, 116 may contain therapeutic nanoparticles. An internal surface of the internal layer 112 may provide slots for attaching multiple sensors strips 120. The sensor strips 120 are configured to monitor different physiological characteristics and may be attached to the slots provided on the internal surface of the patch 110.

The patch 110 may include nanoparticles to provide enhanced therapeutic effects. For example, the nanoparticles may include silver nanoparticles and/or chlorophyll nanoparticles. The patch 110 can be of any shape or size according to the use and area for usage, and may be customized to the size of an ulcer or a wound. For example, the patch 110 may be 21×10 cm.

As described above, the patch 110 may have an internal layer 112, a central layer 114, and an external layer 116. In these embodiments, the internal layer 112 may be made of silicon or may include silicon. The internal silicon layer 112 may be designed to touch the skin of the patient and prevent any adhesion between the patient's skin and the patch 110. Moreover, the internal silicon layer 112 provides potency to the therapeutic effect of the patch 110. The inner layer may also provide slots for fixing the sensor strips 120, which are used to engage and disengage the sensors 122. The internal layer 110 may be porous. The middle layer 114 may also be porous, and in some embodiments, made of sponge or similar material to absorb any excess fluid from excretions. The material of the porous layer can be any porous material that has the same characteristics of a sponge or functions in the same way as a sponge. The external layer 116 may include or be made of silver nanoparticles or chlorophyll nanoparticles. A nano-pad roll 130 may be attached to the external layer 116 of the patch 110. In some embodiments, the nano-pad roll 130 may be wider than the patch 110 it is covering. For example, the nano-pad roll may be 60×20 cm and the patch may be 45×15 cm. The nano-pad roll 130 may be used to encase the patch 110 and a portion of the patient being monitored by the patch 110. For example, the nano-pad roll 130 may be used to encase a diabetic foot ulcer and a patch 110 placed thereon. The nano-pad roll 130 may include or be made of silver nanoparticles or chlorophyll nanoparticles.

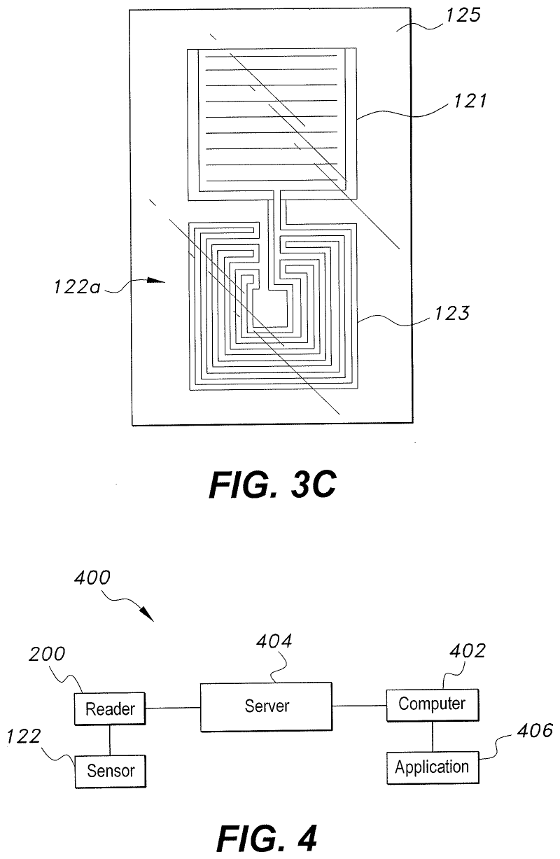

As shown in FIGS. 3A and 3C, one or more of the sensors 122 may be graphene biosensors 122a. The graphene biosensors 122a may include two portions, a microstrip inductive coil 123 and a transducer 121. The transducer 121 may be designed to provide a measurable change in resistance with a measurable change in the physiological characteristic it is designed to monitor. For example, when detecting an amount of bacteria 400, as seen in FIG. 3B, the transducer 121 is designed to attach to select types bacteria 400. The attached bacteria 400 cause an increase in resistance through the transducer 121. When the reader 200 is used to inductively power the graphene biosensor 122a, the increase in resistance can be used to determine the amount of the select bacteria present at the site of the wound or sore.

In some embodiments, as seen in FIG. 3C, the graphene biosensors 122a may be printed on a resorbable substrate 125, such as a silk thin film substrate. The silk thin film substrate 125 provides a medium that can be used to support the biosensor 122a prior to attachment to a sensor strip 120. Once the biosensor 122a is attached to the sensor strip 120, body fluids or added water can be used to dissolve the silk thin film substrate 125, leaving only the graphene biosensor 122a attached to the sensor strip 120.

A basic method of fabricating the graphene biosensor 122a may take place by printing graphene on water-soluble silk thin film substrate 125. Graphene may then be contacted with an interdigitated electrode 121, 123 that is patterned with an inductive coil antenna 123. The structure of graphene-electrode-silk 122a, 125 may then be transferred to a biomaterial (e.g. skin or tissue). Then, functionalization with bifunctional graphene antimicrobial peptides (AMPs) may take place to provide robust bio-recognition moieties. The interdigitated electrode 121, 123 made up of two individually addressable interdigitated comb-like electrode structures may function as an ultra-sensitive electrochemical biosensor.

The fabrication process may start with the growth of monolayer chemical vapor deposition (CVD) graphene on a nickel substrate, etching of the copper, and transfer of graphene through polydimethylsiloxane (PDMS) stamping onto a desired target substrate, which may be the water-soluble silk thin film 125. Then, in order to establish a wireless interrogation, a gold meander line design for the microstrip inductive coil 123 may be deposited on the graphene/silk samples via electron beam evaporation. The graphene sensor 121 with meander line can be connected to an LRC resonant circuit 123 with a gold inductive coil for wireless transmission.

Transferring the biosensor 122a may be accomplished by placing the biosensor 122a on the surface of a biomaterial, such as a tooth or body tissue, within a wet environment whether naturally from the saliva or by applying water moisture to dissolve the silk layer 125. This leaves only the biosensor 122a attached to the desired biomaterial.

Graphene functionalization with AMPs may take place by dissolving bifunctional peptide GBP-OHP (GBP: graphene binding peptide. OHP: AMP odoranin-HP) in deionized (DI) water and dropping it on the transducer 121 of the graphene biosensor 122a, followed by an incubation time of fifteen minutes, washing with deionized water, and then drying to achieve the desired functionalization.

In some embodiments, one of the graphene biosensors 122a may work through monitoring the change in resistance of the transducer 121 functionalized with GBP-OHP when bacteria bind to the immobilized peptides (shown in FIG. 3B). It has been shown that the functionalized graphene with AMP showed a selective behavior of binding to pathogenic bacteria, H. ylori, which is extremely dangerous to human health. Control experiments showed the change in graphene resistance when exposed to H. pylori, which is a Gram-negative pathogen. Similarly, the change in resistance was monitored when the graphene biosensor 122a was exposed to saliva, which is rich in different biomolecules. However, it showed a selective bio-recognition to H. pylori by its binding to the immobilized peptides.

Remote sensing takes place through the use of a single layer thin film inductor-capacitor (LC) resonant circuit 123 integrated in parallel with the resistive graphene transducer 121, which enables wireless readout and battery-free operation. The change in conductance of the graphene sensor 122a when the selected pathogenic bacteria is attached and bonded to the peptides is resolved from changes in the characteristic frequencies and bandwidth of sensor resonance. Both quantities are determined through interaction between the resonant circuit 123 and the reader 200.

A reader device 200 for reading data from a sensor 122 may include a two-turn coil antenna connected to a frequency response analyzer (for example: HP 4191A RF impedance analyzer). The wireless reader 200, which may be powered by an alternating current source, is responsible for wirelessly transmitting power and receiving sensor data from the remote, passive sensor 122 via inductive coupling. Passing an AC signal through the sensor 122 generates a magnetic field, inducing current via mutual inductance in the inductive coil 123 (Faraday's law), and finally resulting in a potential drop that depends on the conductance of the sensor 122. Any change in the conductivity of the sensor 122 resulting from biological or chemical changes occurring at the transducer 121 surface provides a change in the frequency characteristics (namely bandwidth) around the resonance point.

Using a biosensing technique through nanosensors, it is possible to use a biological sensor 122a to detect the type and proportion of bacteria at the site of the wound or the affected area. Embodiments of the biosensors 122a may be designed to detect different signals, such as, but not limited to, bacterial cells, fungi, number of microbial cells, glucose, monosaccharides, disaccharides, polysaccharides, any sugar, glycogen, insulin, hormones, minerals, antibodies, antigens (immunosensor), pH, humidity, temperature, recombinant binding fragments (Fab, Fv or scFv) or domains (VH, VHH) of antibodies, artificial families of Antigen Binding Proteins (AgBP), enzymes, genosensors that detect nucleic acids, epigenetic modifications (e.g. DNA methylation, histone post-translational modifications) in body fluids from patients affected by cancer or other diseases, cancer cells (which may be detected by using photonic biosensors), commonly used organelles including lysosome, chloroplast and mitochondria, cells, stress condition, toxicity, and/or organic derivatives.

The sensor-based healthcare management system may have a patch 110 having slots for attaching or adhering a different number of sensors 122, customized to the need of the home-based patient or following the instructions of a medical professional. The system may have any number of sensors 122 that may be required to detect different conditions of the patient or related to a specific disease. A skilled person in the art can use any number of sensors 122, including biosensors, in the system for sensor-based healthcare management, customized to the needs of the patient.

For example, the medical professional may give instructions to prepare a patch 110 containing a first set of sensors 122 to detect the levels of a specific set of disease-related conditions. The first set of sensors 122 detects the levels of pH, glucose and bacterial species. Upon receiving the medical data transmitted by the first set of sensors 122, the medical professional may give instructions for a first treatment plan. After a period of time, the medical professional can check a second set of disease-related conditions, either in place of or in addition to the first set of disease-related conditions. For example, the second set of disease-related conditions may include bacterial count, humidity, temperature, and GDH enzyme levels. The medial professional may provide instructions related to the patient to prepare and adhere a patch 110 containing sensors 122 for sensing the second set of disease-related conditions. Upon receiving the medical data transmitted by the new set of sensors 122 through the reader 200, which may transmit the data over a network, the medical professional may give instructions for a second treatment plan. In some embodiments, the medical professional may continue to monitor the disease status of the remotely based patient until the complete recovery of the remotely based patient by returning to the healthy state. Embodiments of the present subject matter provide the medical professional with real-time detection of a wide-range of conditions, which comprises a flexible healthcare plan.

In some embodiments of the healthcare management system, the patch 110 may be a therapeutic nano-pad patch that provides a therapeutic effect on the biomaterial or the body-part of the patient. In some embodiments, the therapeutic nano-pad patch 110 is made of or incorporates silver nanoparticles or chlorophyll nanoparticles. In some embodiments, the therapeutic nano-pad patch 110 with silver nanoparticles will continuously release a low level of silver ions to provide protection against microbial cells, e.g., bacteria.

Developing or producing an active agent in the form of nanoparticles provides a potent effect on chemical stability, catalytic activity, physiochemical, and functionalization properties. In some embodiments, the patch 110 may be a therapeutic nano-pad patch 110 of chlorophyll nanoparticles. These embodiments provide chlorophyll nanoparticles, which are a therapeutic composition containing minerals, vitamins and protein extracted from the green material in the plant (clover) prepared in a laboratory and are converted, after the preparation, to the nano size. The chlorophyll nanoparticles may be used with a gel substance and essential nutrients for maintaining the skin vitality and regeneration.

The present system and methods may provide for use in the restoration of skin integrity or acceleration of the restoration of the integrity of an area of a skin or mucosal lesion comprising a broken skin or a damaged mucosa, in part by applying a therapeutic nano-pad patch 110 containing chlorophyll nanoparticles.

The present system and methods provide for a therapeutic nano-pad patch 110 with chlorophyll nanoparticles in the form of a lightweight and user-friendly patch 110. The therapeutic nano-pad patch 110 with chlorophyll nanoparticles has multiple uses, which include, but are not limited to, disinfecting covered tissues from toxins and impurities, healing wounds and burns, resisting wounds bacteria, relieving ulcers and treating some gangrene cases, relieving inflammation pains, reducing secretions, improving thrombocytopenia, and eliminating inflammation and gum bleeding. The chlorophyll nanoparticles have many nutrients and minerals, which include, but are not limited to, zinc, which helps in cell division and growth; calcium, which benefits teeth and bones; potassium, which builds muscle and improves normal growth of the body; phosphorus, which enhances the renal functions and regulates the heartbeat; iron, which contributes to the flow of oxygen in the blood and muscles; vitamin E for skin nutrition; and vitamin C for dental health and gums. The chlorophyll nanoparticles also have anti-carcinogenic properties, vitamin A for strengthening tissues and skin, and in addition, folic acid, magnesium, protein, pantothenic acid, chromium and selenium.

The present system and method may also provide kits for home-based healthcare management for a patient afflicted with a disease. Some embodiments may provide kits for the operation of any agent described herein (e.g., the biosensor 122 attached to therapeutic nano-pad patches 110). Some kits may include an assemblage of materials or components, including at least one of the present sensor-based systems described herein. Thus, in some embodiments, the kit may contain at least one or more sensors 122, patches 110, and/or rolls 130.

Some embodiments of the present system and method provide for a kit, which comprises different color-coded sensors 122. The color-coded sensors 122 can be easily distinguished and handled by the elderly and also people with disabilities. Some kits may include plastic strips 124 for attachment of any number of sensors to the patch, as seen in FIG. 1. The plastic strips 124 are easy to handle for engaging and disengaging the sensors 122 with the patch 110. Some embodiments of the kits may include alcohol pads or a solution to disinfect the plastic strips 124 for re-use with different sensors 122.

Some embodiments of the sensor-base healthcare management system include a reader 200 to receive the signals from the sensors 122 and translate it to a medical data of the different disease-related conditions. In addition, the reader 200 may provide a magnetic field to induce a current within the circuit through inductive coupling. The reader 200 may then transmit the medical data to a medical professional over a network. The reader device can be, but is not limited to, a mobile, laptop, computer, or any device of the same function herewith.

The exact nature of the components included in the kit depends on the intended purpose of the kit. For example, in one embodiment, the kit may be configured for the purpose of treating human subjects. The kit may have separated components so it provides the flexibility to pick, choose, and change any component to accommodate the instruction or request of the medical professional or the person in charge. In some embodiments, the kit may be portable. Accordingly, the system and methods for sensor-based healthcare management can be used and provided at home, work, hospital, medical facility, or any building. Some kits may be lightweight, easy to use, safe for adults and children, and have multi-use as different sensors 122 can detect disease-related conditions of different disorders, e.g., teeth inflammations, diabetic ulcers, wounds, ulcers and gangrene cases.

Instructions for use may be included in the kit (i.e., a booklet). Instructions for use typically include a tangible expression describing the technique to be employed in using the components of the kit to effect a desired outcome, such as to treat diabetic foot ulcer. Optionally, the kit may also contain other useful components, such as, diluents, buffers, pharmaceutically acceptable carriers, syringes, catheters, applicators, pipetting or measuring tools, bandaging materials or other useful paraphernalia, as will be readily recognized by those of skill in the art.

The components within a kit are typically contained in suitable packaging materials. The packaging material can be in the form of, but not limited to, a bag or a box, or any container of the same function. In various embodiments, the packaging material is constructed by well-known methods, preferably to provide a sterile, contaminant-free environment. The packaging material may have an external label, which indicates the contents and/or purpose of the kit and/or its components.

Some embodiments of the sensor-based healthcare management methods and system described herein have applications of treating or preventing various diseases and disorders, including, but not limited to, cancer, infections, immune disorders, anemia, autoimmune diseases, cardiovascular diseases, wound healing, deep wounds, surgical-operations wounds, ischemia-related diseases, neurodegenerative diseases, metabolic diseases and many other diseases and disorders. In some embodiments, the sensor-based healthcare management methods and system may be configured for the treatment of a patient having a microbial infection and/or chronic infection. Illustrative infections include, but are not limited to, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections. In various embodiments, the healthcare management systems and methods may be used to treat wounds, e.g., a non-healing wound, an ulcer, a burn, or frostbite, a chronic or acute wound, open or closed wound, internal or external wound (illustrative external wounds are penetrating and non-penetrating wound). In one or more embodiments, the disease or disorder is selected from the group consisting of a wound, a chronic wound, a burn, impetigo, acne, rosacea, an inflammation, an ulcer, and a skin disease caused by bacteria. In some embodiments, the disorder may be a wound, a chronic wound, impetigo, acne, rosacea, an inflammation, a skin disease caused by a bacteria, a skin disease caused by a fungus, a skin disease caused by a virus, a diabetic foot ulcers, gangrene, and tooth inflammation.

The sensor-based healthcare management methods and system may provide for treating or preventing damaged skin ulcer diseases, including mechanical trauma, burns, pressure sores, chronic leg ulcers, diabetic foot ulcer or cancer. In some embodiments, the skin ulcer is located on the upper extremity. In some embodiments, the skin ulcer is located on the hand or fingers. In other embodiments, the skin ulcer is located on the lower extremity. In some embodiments, the skin ulcer is located on the foot. In some embodiments, the skin ulcer is caused by arterial insufficiency, venous insufficiency, lymphatic insufficiency, pressure, neuropathy, trauma or a combination thereof. In other embodiments, the skin ulcer is caused by a metabolic, inflammatory, infectious, neoplastic, degenerative or hereditary disease or a combination thereof. In some embodiments, the skin ulcer is caused by diabetes. In other embodiments, the skin ulcer is caused by atherosclerosis. In other embodiments, the pressure sores results from immobility, paralysis or obesity of the patient.

A method for treating or preventing a disease afflicting a home-based patient, may comprise using a sensor-based system adhered to a specific body-part of the patient to detect at least one condition of a disease indicative of a status of the disease. The method may include positioning a sensor-based system 10 including a first set of sensors 122 over an area to be treated for a disease or condition; collecting medical data about the at least one disease condition; transmitting the medical data collected to a receiving system for analysis and notifying a medical professional; reviewing the medical data received by the medical professional and determining that medical data indicates a first status of the patient; receiving instructions for a first medical plan from the medical professional by the patient; administering an effective dose of medication, as a first medical plan, as instructed by the medical professional; collect levels of the at least one condition of the disease by the sensor-based system after a time period and determining if a second medical plan is required as determined by the medical professional; determining if a second set of sensors 122 are needed to detect new conditions of the disease as required by the medical professional, and repeating the previous steps till the patient treatment or prevention from the disease is achieved, as revealed by the medical data collected by the sensor-based system for the at least one condition of the disease and the new conditions of the disease.

The embodiments of the present subject matter provide systems, methods, and kits that prevent, treat, and heal diabetic ulcers. In these embodiments, the present system has the flexibility to detect different disease conditions causing and complicating diabetic ulcers through a set′ of sensors 122, which may be biosensors made of graphene. The present system further provides for a nano-pad patch 110, which has a potent therapeutic effect on diabetic ulcers and helps to prevent, treat, and heal it. A nano-pad roll 130 may cover the patch 110 and encases the diabetic ulcer. Collectively, the patient receives real-time monitoring of any diabetic-ulcer-related condition (or biomarker), which helps the medical professional to track the disease state and prescribe suitable medication, and additionally, the patient receives the potent therapeutic effect of the nano-pad patch 110 and roll, that helps to expedite the treatment and healing of the diabetic ulcer (in addition to medication). In some embodiments, the therapeutic effect of the nano-pad patch 110 and roll 130 is due to the silver nanoparticles. In other embodiments, the therapeutic effect of the nano-pad patch 110 and roll 130 is due to the chlorophyll nanoparticles.

The present system provides systems and methods, which are suitable for patients with chronic cases, as well as the elderly who stay in bed for a long time and that need continuous follow-up, and find it difficult to go every day to the hospital to monitor their wound, take ulcer biopsies for examination, and see the results of periodic analysis. Additionally, embodiments of the present subject matter may also provide early detection of inflammation and severity the wound or sores by taking into account the precise monitoring of ulcers and deep and chronic wounds. In the case of any signs of inflammation detected by the sensor, a treatment plan can be prescribed immediately.

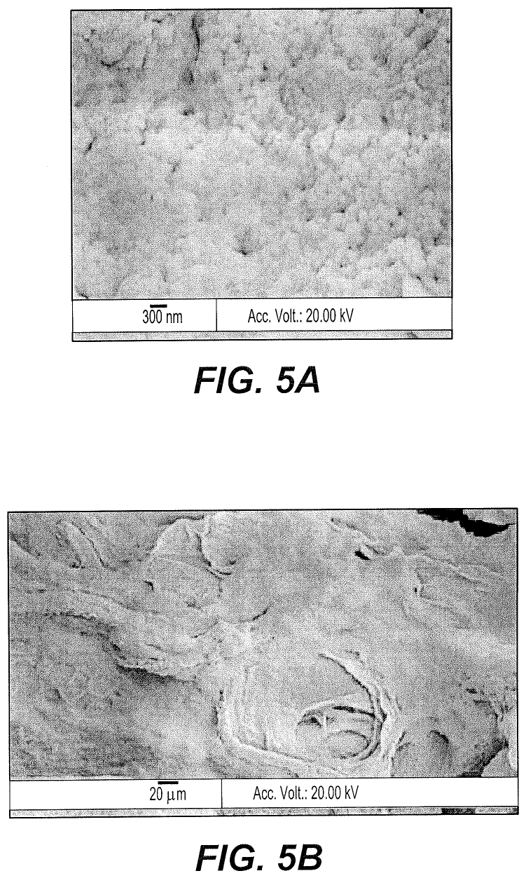

FIG. 4 shows a diagram of an embodiment of a network used by the system 400. The sensor 122 communicates with the reader 200. As previously discussed, the reader 200 may inductively couple with the sensor 122 to provide the sensor 122 with the power for transmitting a wireless signal to the reader 200, similar to an RFID system. The reader 200 communicates with a server 400. Communication between the reader 200 and the server 404 may be through wireless and wired pathways known in the art for transmitting data, such as, but not limited to, Ethernet, Wi-Fi, cellular communications, or a combination thereof. In some embodiments, the reader 200 may communicate with a computer at the location of the reader 200, which communicated with the server 404. The server 404 communicates with a computer 402 at a remote location. Communication between the server 404 and the computer 402 may be through wireless and wired pathways known in the art for transmitting data, such as Ethernet, Wi-Fi, cellular communications, or a combination thereof. The information is presented in usable form by an application 406 on the computer 402. A practitioner may receive data or medical information on the application 406 from the sensors 122 and then send instructions back to the reader 200 via the application 406 based on the received information. A patient or patient care technician can use the information sent to the reader to alter the medical plan and/or sensors 122.

### Example 1

### Extraction of Chlorophyll Nanoparticles

The process starts by the selection of an intact white clover plant (Alfalfa) that is free of lesions and diseases, as it is a large source of chlorophyll (this plant has been cultivated at the fields of the University of Kuwait).

The steps for extracting chlorophyll include the following. (1) Separate the leaves, avoiding the old lower legs of the plant and damaged leaves, and then put the undamaged leaves in a Petri-dish; (2) Clean the leaves thoroughly with water; (3) Put the leaves of the plant in a mortar and crush the leaves with a pestle to obtain a mixture of leaves; (4) Filter the part of the liquid mixture of the leaves using gauze made of sterile cloth; (5) The extract obtained is green, deep, and turbid, with the solid particles remaining in the cloth; (6) Place the filtered syrup of chlorophyll protein in a pot and heat it at boiling temperature for 5 minutes, which presents the chlorophyll proteins held by the extract; (7) Purify the chlorophyll, pressing on it to compress the excess liquid while ignoring the reddish brown liquid; (8) Wash chlorophyll crystals gently with cold water and filter them through sterile cloth or medical gauze; this process will remove a large part of the red dye soluble in water so the powder will be bright green; (9) Spread the wet chlorophyll in a very thin layer on the wax paper then put it in oven until it is dry, which may take 1-2 hours, or in some cases, up to 50 hours; the oven should be open slightly to allow moisture to escape; (10) Remove the dry extract from the wax paper once the drying process is complete; (11) Grind the dry extract in a grinding device to obtain chlorophyll powder containing chlorophyll A, B, C, and D, and using a sieve to obtain a nano-powder with particles having a diameter in the range of 75-100 nm;

### Example 2

### Testing of Chlorophyll Nanoparticles

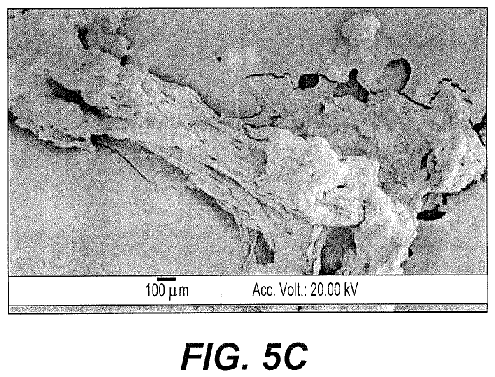

The chlorophyll nanoparticles, including chlorophyll A, B, C, and D, prepared as in Example 1, have been examined at the University of Kuwait's Nanoscopy Center using a scanning electron microscope (SEM) and Scanning Tunneling Microscopy (STM). The product is shown in FIGS. 5A-5C, which indicates the size of the nanoparticles are primarily in the range of 75 to 100 nm.

For preparation of chlorophyll nanoparticles in a nano-pad 110, the chlorophyll nanoparticles are mixed with a gelatin substance and nutrients for maintaining skin vitality and regeneration.

An experiment was done at the Kuwait Institute for Scientific Research, Department of Microbiology to show the therapeutic effect of chlorophyll nanoparticles on eliminating infectious bacteria. Two types of bacterial cultures were examined, viz., Staphylococcus aureus and pseudomonas sp. Superior results were obtained that show a promising bacterial therapeutic effect of the chlorophyll nanoparticles.

Another set of experiments was conducted in a veterinary hospital in Kuwait on a group of rabbits to show the therapeutic effect of the chlorophyll on wounds and ulcers. The rabbits were cured from wounds and ulcers within five to seven days of using the sterile gauze saturated with chlorophyll nanoparticles. The results were satisfactory and the experiments were conducted under the supervision of a veterinarian.

It is to be understood that the system and method for sensor-based healthcare management is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A system for measuring a specific set of disease-related conditions comprising:
a patch adapted for attachment to a body part of a patient at a site designated by a health care professional, wherein the patch comprises an internal layer adapted for direct contact with the body part of the patient, an external layer facing away from the body part, and a porous layer disposed between the internal layer and the external layer;
a set of biosensors selectively attachable to the internal layer of the patch, each of the biosensors being configured for detecting a distinct disease-related condition and generating an electrical signal corresponding to the disease-related condition when the disease-related condition is detected, the set of biosensors including at least:
a) pH sensor;
b) moisture sensor;
c) temperature sensor;
d) microbial infection sensor; and
e) graphene sensor;
a set of transmitter circuits, each of the biosensors having a corresponding one of the circuits connected thereto, each of the circuits being configured for transmitting a radio frequency signal at a transmitter frequency modulated by the electrical signal generated by the corresponding biosensor; and
a reader configured for periodically polling the transmitter circuits and receiving the modulated radio frequency signals at the transmitter frequency.

2. The system for sensor-based healthcare management according to claim 1, further comprising a server connected to the reader, the server being connected to a communications network, the server being configured for converting the modulated radio frequency signals received by the reader into medical data and automatically forwarding the medical data to the health care professional in real time, whereby the health care professional may remotely monitor treatment rendered to the patient.

3. (canceled)

4. The system for sensor-based healthcare management according to claim 1, further comprising a plurality of strips selectively attached to said patch, each of said biosensors being rigidly attached to a corresponding one of the strips, whereby individual biosensors in said set of biosensors may be selectively removed and replaced by a biosensor configured for detecting a different disease-related condition.

5. The system for sensor-based healthcare management according to claim 4, wherein each said strip is color-coded according to the disease-related condition the corresponding biosensor is configured to detect.

6. (canceled)

7. The system for sensor-based healthcare management according to claim 1, wherein the at least one of said biosensors comprises a graphene biosensor having comprises a graphene trace configured as one of the transmitter circuits, the graphene being functionalized to detect the disease-related condition.

8. The system for sensor-based healthcare management according to claim 1, wherein each said transmitter circuit includes a microstrip induction coil, whereby said transmitter circuits receive power for transmitting inductively from polling signals from said reader.

9. The system for sensor-based healthcare management according to claim 1, further comprising a roll wrapping said patch around the body part of the patient.

10. The system for sensor-based healthcare management according to claim 1, further comprising a nanoparticle treatment agent incorporated into said patch for gradual release at the site designated by the health care professional.

11. The system for sensor-based healthcare management according to claim 10, wherein said nanoparticle treatment agent comprises silver nanoparticles.

12. The system for sensor-based healthcare management according to claim 10, wherein said nanoparticle treatment agent comprises chlorophyll nanoparticles.

13. The system for sensor-based healthcare management according to claim 10, wherein said nanoparticle treatment agent comprises nanoparticles of an extract from leaves of clover plants.

14. A method for sensor-based healthcare management, comprising the steps of:
maintaining a patch having a plurality of interchangeable biosensors selectively attached to a body part of a patient, each of the biosensors being configured for detecting a distinct disease-related condition, each of the biosensors being connected to a transmitter circuit for transmitting a signal modulating output from the biosensor;
automatically periodically polling the biosensors using a reader to obtain output from each of the biosensors, the reader being connected to a communications network; and
automatically communicating the output from each of the biosensors to a health care professional over the communications network so that the health care professional may monitor a patient's condition remotely in real time.

15. The method for sensor-based healthcare management according to claim 14, further comprising the step of treating the patient's condition with therapeutic nanoparticles incorporated into the patch for gradual release into the body part of the patient.

16. The method for sensor-based healthcare management according to claim 15, wherein the patient's condition comprises diabetic foot ulcers and the therapeutic nanoparticles comprises nanoparticles of chlorophyll extracted from leaves of clover plants

17. The method for sensor-based healthcare management according to claim 14, further comprising the step of replacing at least one of the biosensors with a biosensor configured for detecting a different disease-related condition as needed, based upon the output of the biosensors.

18. The method for sensor-based healthcare management according to claim 17, wherein each said biosensor is mounted on a corresponding color-coded plastic strip, the color-coded plastic strip being selectively and removably attached to said patch, said step of replacing at least one of the biosensors comprising removing one of the color-coded plastic strips from said patch and replacing the removed strip with a color-coded plastic strip of a different color corresponding to the desired biosensor.

19. The method for sensor-based healthcare management according to claim 14, wherein said step of maintaining a patch having a plurality of interchangeable biosensors selectively attached to a body part of a patient comprises wrapping a roll bandage around the patch and the body part.

20. A kit for measuring a specific set of disease-related conditions, comprising:
a patch adapted for attachment to a body part of a patient at a site designated by a health care professional, wherein the patch comprises an internal layer adapted for direct contact with the body part of the patient, an external layer facing away from the body part, and a porous layer disposed between the internal layer and the external layer;
a set of biosensors selectively attachable to the internal layer of the patch, each of the biosensors being configured for detecting a distinct disease-related condition and generating an electrical signal corresponding to the disease-related condition when the disease-related condition is detected, the set of biosensors including at least:
a) pH sensor;
b) moisture sensor;
c) temperature sensor;
d) microbial infection sensor; and
e) graphene sensor;
a set of transmitter circuits, each of the biosensors having a corresponding one of the circuits connected thereto, each of the circuits being configured for transmitting a radio frequency signal at a transmitter frequency modulated by the electrical signal generated by the corresponding biosensor, each of the transmitter circuits being mounted on the color-coded plastic strip with the corresponding biosensor;
a roll bandage adapted for attaching the patch to the body part of a patient and
a reader configured for periodically polling the transmitter circuits and receiving the modulated radio frequency signals at the transmitter frequency, the reader being connectable to a communications network for forwarding the signals from the biosensors to a monitoring health care professional in real time.
